(19) United States
(12) Patent Application Publication
(10) Pub. No.: US 2025/0201005 A1
(43) Pub. Date: Jun. 19, 2025

(54) DEDUPLICATION OF SAMPLE IMAGES

(71) Applicant: GENENTECH, INC., South San Francisco, CA (US)

(72) Inventors: Xingyue GE, Millbrae, CA (US); Umme Salma Yusuf BENGALI, Sunyvale, CA (US); Noel Andres CARRASCAL, San Francisco, CA (US)

(21) Appl. No.: 19/072,454

(22) Filed: Mar. 6, 2025

Related U.S. Application Data
(63) Continuation of application No. PCT/US2023/073742, filed on Sep. 8, 2023.
(60) Provisional application No. 63/405,332, filed on Sep. 9, 2022.

(51) Int. Cl. G06V 20/69; G06T 3/40; G06T 7/11; G16H 30/20; G16H 30/40

(52) U.S. Cl. CPC G06V 20/698; G06T 3/40; G06T 7/11; G16H 30/20; G16H 30/40

(57) ABSTRACT

A method includes identifying, based at least on an identification information associated with an image set depicting one or more slices of tissue from at least one block of a tissue sample, one or more of a duplicate image, a replicate image, and a multiple image present within the image set. The one or more of the duplicate image, the replicate image, and the multiple image may be further identified based on a metric indicative of a minimal difference achieved between each pair of images in the image set while adjusting an alignment therebetween. The identification information associated with the image set may be updated to indicate the one or more of the duplicate image, the replicate image, and the multiple image identified as present within the image set. Related systems and computer program products are also disclosed.

### CROSS REFERENCE TO RELATED APPLICATION

This application claims priority to U.S. Provisional Application No. 63/405,332, filed on Sep. 9, 2023 and entitled “DEDUPLICATION OF SAMPLE IMAGES,” the disclosure of which is incorporated herein by reference in its entirety.

### FIELD

The present disclosure generally relates to digital pathology and more specifically to the deduplication of sample images.

### INTRODUCTION

In digital pathology, whole slide images of tissue samples can be used for computational processes. In many instances, sets of the whole slide images contain either identical or similar copies of digital or physical samples. Such copies are generally removed before the set of whole slide images is used during the computational processes. However, removing the copies can be cumbersome, inefficient, and take a long time. Further complicating the removal of the copies, it can be difficult to identify which images from the set of images are copies. For example, many images may be associated with mismatched information, have incorrect labels, and/or have missing information. Many images may also be similar to one another, but not identical. Thus, it can be difficult to easily and quickly identify the copies so that they can be removed for use in the computational processes.

### SUMMARY

Methods, systems, and articles of manufacture, including computer program products, are provided for deduplication of sample images. In one aspect, there is provided a system. The system may include at least one processor and at least one memory. The at least one memory may store instructions that result in operations when executed by the at least one processor. The operations may include: identifying, based at least on an identification information associated with a plurality of images depicting one or more slices of tissue from at least one block of a tissue sample, one or more of a duplicate image, a replicate image, and a multiple image present within the plurality of images; identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and updating the identification information associated with the plurality of images to indicate the one or more of the duplicate image, the replicate image, and the multiple image identified as present within the plurality of images.

In another aspect, there is provided a method for identifying duplicate images. The method may include: identifying, based at least on an identification information associated with a plurality of images depicting one or more slices of tissue from at least one block of a tissue sample, one or more of a duplicate image, a replicate image, and a multiple image present within the plurality of images; identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and updating the identification information associated with the plurality of images to indicate the one or more of the duplicate image, the replicate image, and the multiple image identified as present within the plurality of images.

In another aspect, there is provided a computer program product that includes a non-transitory computer readable storage medium. The non-transitory computer-readable storage medium may include program code that causes operations when executed by at least one processor. The operations may include: identifying, based at least on an identification information associated with a plurality of images depicting one or more slices of tissue from at least one block of a tissue sample, one or more of a duplicate image, a replicate image, and a multiple image present within the plurality of images; identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and updating the identification information associated with the plurality of images to indicate the one or more of the duplicate image, the replicate image, and the multiple image identified as present within the plurality of images.

In another aspect, there is provided an apparatus that includes: means for identifying, based at least on an identification information associated with a plurality of images depicting one or more slices of tissue from at least one block of a tissue sample, one or more of a duplicate image, a replicate image, and a multiple image present within the plurality of images; means for identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and means for updating the identification information associated with the plurality of images to indicate the one or more of the duplicate image, the replicate image, and the multiple image identified as present within the plurality of images.

In some variations of the methods, systems, and non-transitory computer readable media, one or more of the following features can optionally be included in any feasible combination.

In some variations, the identification information may include at least a portion of a manifest, a scannable barcode, and/or metadata associated with the plurality of images.

In some variations, the identification information may include one or more of a sample identifier, a patient identifier, a block identifier, a slide identifier, an imaging modality, and a scanning parameter.

In some variations, a first image of the plurality of images may be identified as a duplicate of a second image of the plurality of images based at least on the first image and the second image being associated with matching sample identifiers, patient identifiers, block identifiers, and slide identifiers.

In some variations, a first image of the plurality of images may be identified as a replicate of a second image of the plurality of images based at least on the first image and the second image being associated with matching sample identifiers, patient identifiers, and block identifiers but different slide identifiers.

In some variations, a first image of the plurality of images may be identified as a multiple of a second image of the plurality of images based at least on the first image and the second image being associated with matching sample identifiers and patient identifiers but different block identifiers and different slide identifiers.

In some variations, the identification information may be updated by at least including, in the identification information, a flag indicating the one or more of the duplicate image, the replicate image, and the multiple image present within the plurality of images.

In some variations, the minimal difference between each pair of images may correspond to a minimal difference between a first vector field of a first image included in each pair of images and a second vector field of a second image included in each pair images.

In some variations, the first vector field and the second vector field may be generated. The difference between the first vector field and the second vector field may be determined while adjusting the alignment between the first image and the second image until achieving the minimal difference between the first vector field and the second vector field.

In some variations, each of the first vector field and the second vector field may include a plurality of vectors. Each vector of the plurality of vectors may correspond to a section within a corresponding image. Each vector of the plurality of vectors may have a direction and a magnitude corresponding to a change in an intensity values of one or more pixels included a corresponding section of the corresponding image.

In some variations, each of the first image and the second image may be divided into a plurality of sections. A size of each section of the plurality of sections and/or a quantity of the plurality of sections may be adjusted until the minimal difference between the first vector field and the second vector field is achieved.

In some variations, the alignment between the first image and the second image may be adjusted by at least translating, rotating, and/or inverting the first image relative to the second image.

In some variations, the difference between the first vector field and the second vector field may be determined by at least subtracting one or more x-components of the first vector field and the second vector field separately from one or more y-components of the first vector field and the second vector field.

In some variations, a first image in each pair of images may be identified as a duplicate of a second image in each pair of images based at least on the metric satisfying a first threshold.

In some variations, the first image may be identified as a replicate of the second image based at least on the metric failing to satisfy the first threshold but satisfying a second threshold greater than the first threshold.

In some variations, the first image and the second image may be identified as adjacent images from a same block of the tissue sample based at least on the metric satisfying the second threshold. The identification information associated with the plurality of images may be updated to indicate the first image and the second image as adjacent images from the same block of the tissue sample.

In some variations, the first image and the second image may be converted into grayscale images prior to generating the first vector field and the second vector field.

In some variations, a scale of pixel intensity values in each of the first image and the second image may be converted from a first scale to a second scale prior to generating the first vector field and the second vector field.

In some variations, the first scale may be [0, 1] and the second scale may be [−1, 1].

In some variations, the one or more of the duplicate image, the replicate image, and the multiple image present within the plurality of images may be first identified based on the identification information before the metric is computed for a remaining plurality of images to identify the one or more of the duplicate image, the replicate images, and the multiple images in the remaining plurality of images.

In some variations, at least one duplicate image present within the plurality of images may be deleted.

In some variations, the identification information may be updated to correct at least one discrepancy between the identification information and the one or more of the duplicate image, the replicate image, and the multiple image identified based on the metric.

In some variations, a sequence of the plurality of images indicated in the identification information may be updated to restore, based at least on an ordering of at least one replicate image identified within the plurality of images, an original sequence of images within the at least one block of the tissue sample.

In some variations, a sequence of the plurality of images indicated in the identification information may be updated to restore, based at least on an ordering of at least one multiple image identified within the plurality of images, an original sequence of images across different blocks of the tissue sample.

In some variations, the duplicate image may depict a same slice of tissue as another image included in the plurality of images. The replicate image may depict a different slice of tissue from a same block of the tissue sample as the another image included in the plurality of images. The multiple image may depict a slice of tissue from a different block of the tissue sample as the another image from the plurality of images.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to whole slide images, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts an example sample image deduplication system, consistent with implementations of the current subject matter;

FIG. 2 depicts an example tissue specimen and tissue slide, consistent with implementations of the current subject matter;

FIGS. 3A and 3B depict a comparison between example tissue samples, consistent with implementations of the current subject matter;

FIG. 4 depicts an example implementation of a tool of a sample image deduplication system, consistent with implementations of the current subject matter;

FIG. 5 depicts an example of a manifest, consistent with implementations of the current subject matter;

FIG. 6 depicts an example of a manifest, consistent with implementations of the current subject matter;

FIGS. 7A and 7B illustrate example barcodes of a tissue sample slide, consistent with implementations of the current subject matter;

FIG. 8A illustrates examples of vector fields, consistent with implementations of the current subject matter;

FIG. 8B illustrate an example of a quadrant of four pixels from an image, consistent with implementations of the current subject matter;

FIG. 8C illustrate another example of a quadrant of four pixels from an image, consistent with implementations of the current subject matter;

FIG. 8D illustrates an example of vector fields, consistent with implementations of the current subject matter;

FIG. 8E illustrates another example of vector fields, consistent with implementations of the current subject matter;

FIG. 9 illustrates an example vector field plot, consistent with implementations of the current subject matter;

FIG. 10A depicts a flowchart illustrating an example of a process for deduplicating sample images, consistent with implementations of the current subject matter;

FIG. 10B depicts a flowchart illustrating another example of a process for deduplicating sample images, consistent with implementations of the current subject matter;

FIG. 11 depicts a flowchart illustrating an example of a process for identifying duplicate, replicate, and/or multiple images, consistent with implementations of the current subject matter;

FIG. 12 depicts a flowchart illustrating an example of a process for identifying duplicate, replicate, and/or multiple images, consistent with implementations of the current subject matter;

FIG. 13 depicts a flowchart illustrating an example of a process for identifying duplicate, replicate, and/or multiple images, consistent with implementations of the current subject matter; and

FIG. 14 depicts a block diagram illustrating an example of a computing system, consistent with implementations of the current subject matter.

When practical, like labels are used to refer to same or similar items in the drawings.

### DETAILED DESCRIPTION

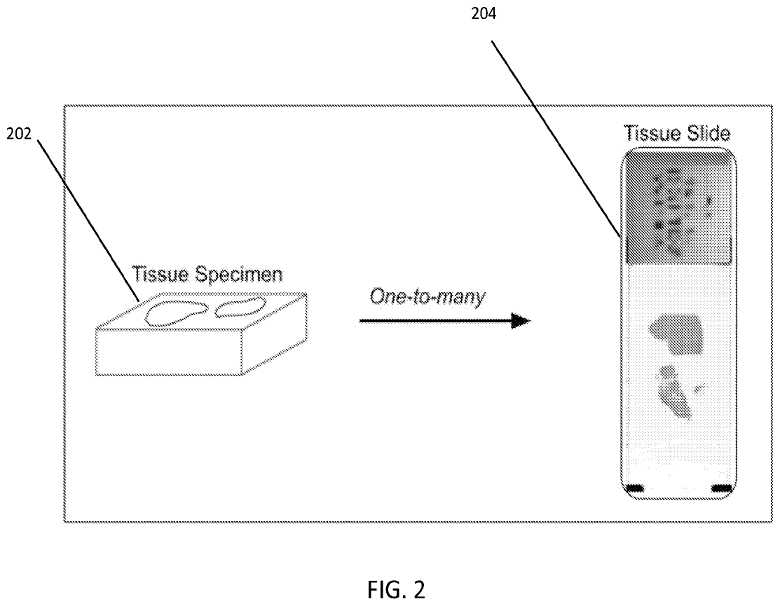

In digital pathology, whole slide images of tissue slides can be used for computational processes. FIG. 2 shows an example tissue specimen 202, which includes tissue from an organ of a patient. Further referring to FIG. 2, a tissue slide 204 may contain a slice of the tissue specimen 202. The tissue slide 204 may also include an identifier, such as a barcode including identification information associated with the tissue slide. The identification information may indicate the origin of the tissue specimen 202 included in the tissue slide 204. For example, the identification information may include an identifier associated with a sample, a block, and/or a slice of the tissue specimen 202. As described herein, a sample refers to a tissue specimen (e.g., the tissue specimen 202) taken from a particular patient. Each sample may include at least one block of the tissue specimen. For example, each sample includes a plurality of adjacent blocks of the tissue specimen that were retrieved from an organ of the patient. Each block of the tissue specimen may include a slice. As described herein, a slice refers to a section of the tissue specimen for a particular block. Thus, the tissue slide 204 includes a slice of a block of a tissue sample.

The whole slide images can be generated by capturing and storing images of a whole slide, such as the tissue slide 204. As noted above, sets of the whole slide images can be used for different computational purposes, such as in machine learning, artificial intelligence, or other systems, or computational processes. However, the sets of whole slide images may contain duplicates, replicates, or multiples, reducing the accuracy and efficiency of the computational processes.

Consistent with implementations of the current subject matter, duplicate images are images of the same tissue slides that are from the same block of a tissue specimen of a patient. Duplicate images may include identical copies or images that include identical or close to identical (e.g., captured from a different focal point, modality, etc.). Consistent with implementations of the current subject matter, replicate images are images of tissue slides including tissue from the same block, but are different slices of the tissue. Replicate images may include serial sections or slices related to one another that are similar in appearance, but are not the same. Replicate images may also include slices that are intact or disrupted (e.g., cut) contiguous samples, missing tissue, have floating tissue, or the like. Consistent with implementations of the current subject matter, multiples are images of tissue slides that contain tissue samples from the same patient, but are from different blocks. Images that are duplicates, replicates, and/or multiples can be related to one another such that they are not considered to represent independent data points during the computational processes. Inclusion of duplicate images, replicate images, and/or multiples in a set of whole slide images for use with computational processes can be detrimental to the performance of those computational processes.

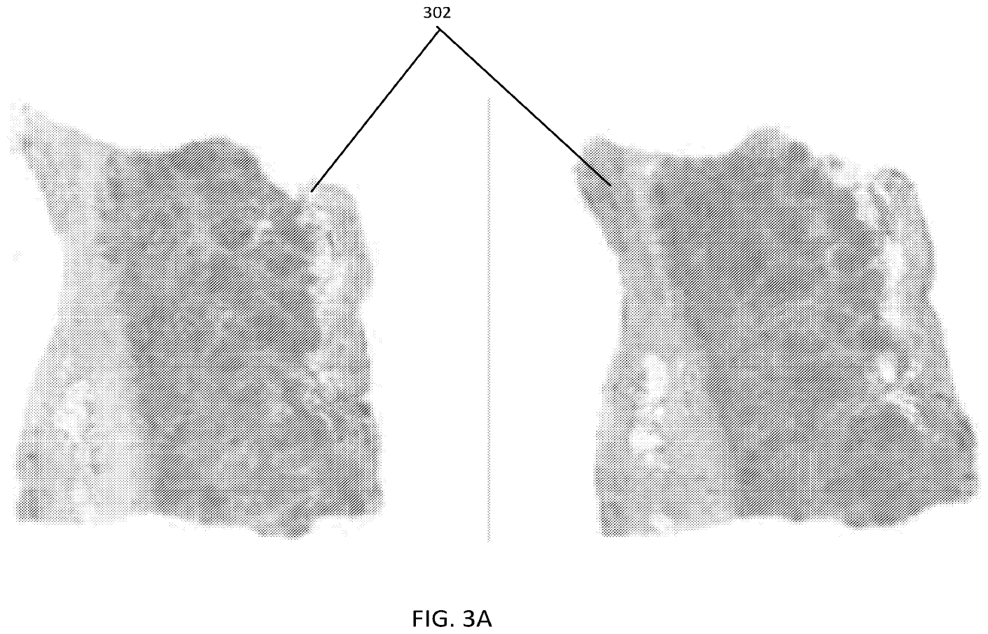

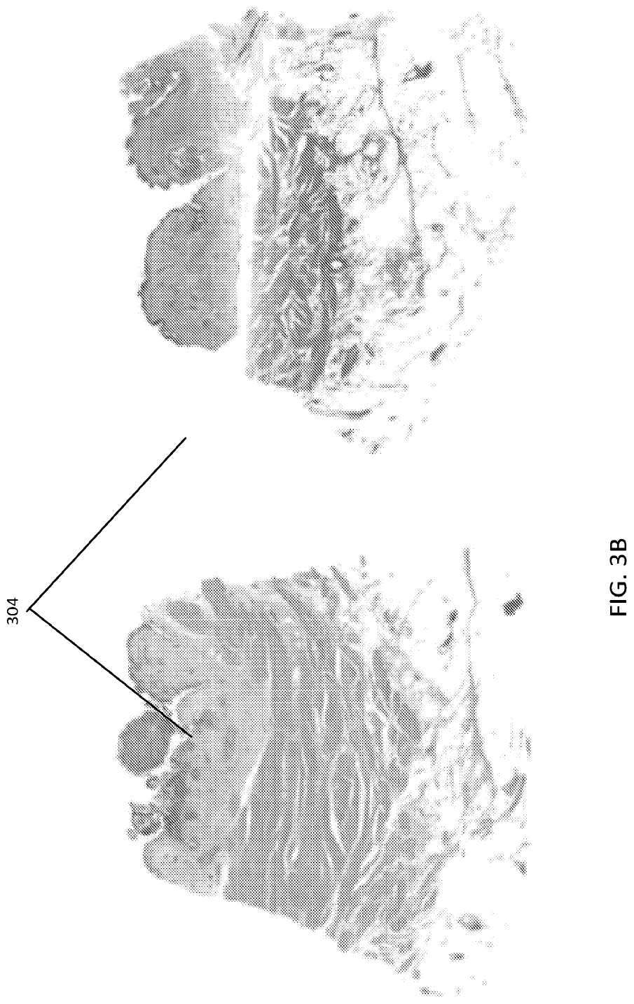

To improve the performance of the computational processes, duplicate images, replicate images, and/or multiples may be removed from the set of whole slide images can be removed. However, removing the copies can be cumbersome, inefficient, and take a long time. Further complicating the removal of the copies, it can be difficult to identify which images from the set of images are copies. For example, many images may be associated with mismatched information, have incorrect labels, and/or have missing information. Many images may also be similar to one another, but not identical. For example, FIGS. 3A and 3B each show side-by-side images of tissue samples 302, 304 taken from the same block. As shown in FIGS. 3A and 3B, it can be difficult to identify images include similar samples. Thus, it can be difficult to easily and quickly identify the duplicate images, replicate images, and/or multiples for removal.

The deduplication system consistent with implementations of the current subject matter may identify duplicates, replicates, and/or multiples in sets of whole slide images for removal from the image data sets using an automated tool and/or image processing techniques that are computationally cheaper, quicker, and more efficient than conventional techniques. For example, the deduplication system includes a deduplication engine that compares information, such as data related to the images stored in a manifest and/or data embedded in the images, and based on the information, identifies the duplicates, replicates, and/or multiples. The deduplication engine may also generate a metric based on a comparison of remaining images not previously identified as a duplicate, replicate, and/or multiple, and based on the metric, predict whether the remaining images are duplicates, replicates, and/or multiples. Based on the identification and/or the prediction, the duplicate, replicate, and/or multiple images may be removed from the set of the whole slide images, such as for use in the computational processes.

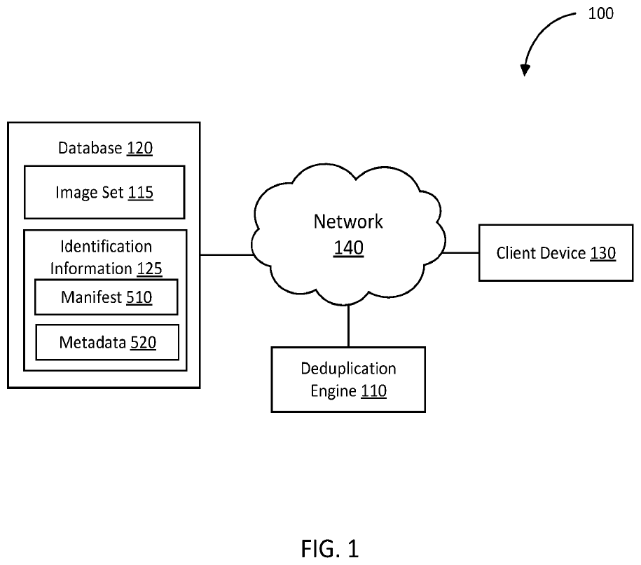

FIG. 1 depicts a system diagram illustrating an example of a deduplication system 100, consistent with implementations of the current subject matter. Referring to FIG. 1, the deduplication system 100 may include a deduplication engine 110, a client device 130, and a database 120. The deduplication engine 110, the database 120, and the client device 130 may be communicatively coupled via a network 140. The network 140 may be a wired network and/or a wireless network including, for example, a wide area network (WAN), a local area network (LAN), a virtual local area network (VLAN), a public land mobile network (PLMN), the Internet, and/or the like. In some implementations, the deduplication engine 110, the database 120, and/or the client device 130 may be contained within and/or operate on a same device. It should be appreciated that the client device 130 may be a processor-based device including, for example, a smartphone, a tablet computer, a wearable apparatus, a virtual assistant, an Internet-of-Things (IoT) appliance, and/or the like. The deduplication engine 110 may include at least one processor and at least one memory storing instructions which, when executed by the at least one processor, perform one or more operations.

In some example embodiments, the deduplication engine 110 may be configured to identify, within an image set 115, one or more of a duplicate image, a replicate image, and a multiple image. The image set 115 may include a plurality of images depicting one or more slices of tissue from at least one block of a tissue sample. As used herein, the term “duplicate image” may refer to an image depicting a same slice of tissue as another image included in the image set 115. The term “replicate image” may refer to an image depicting a different slice of tissue from a same block of the tissue sample as the another image included in the image set 115. The term “multiple image” may refer to an image depicting a slice of tissue from a different block of the tissue sample as the another image from the image set 115. In the example shown in FIG. 1, the image set 115 may be a set of whole slide images stored, for example, in the database 120 in any format and in varying resolutions.

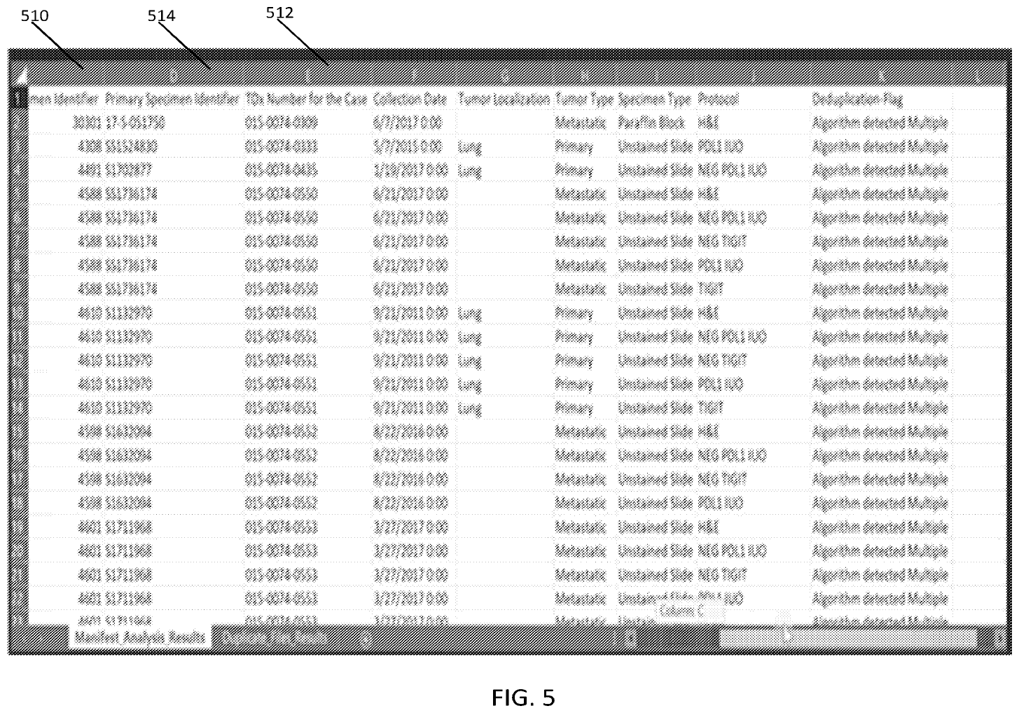

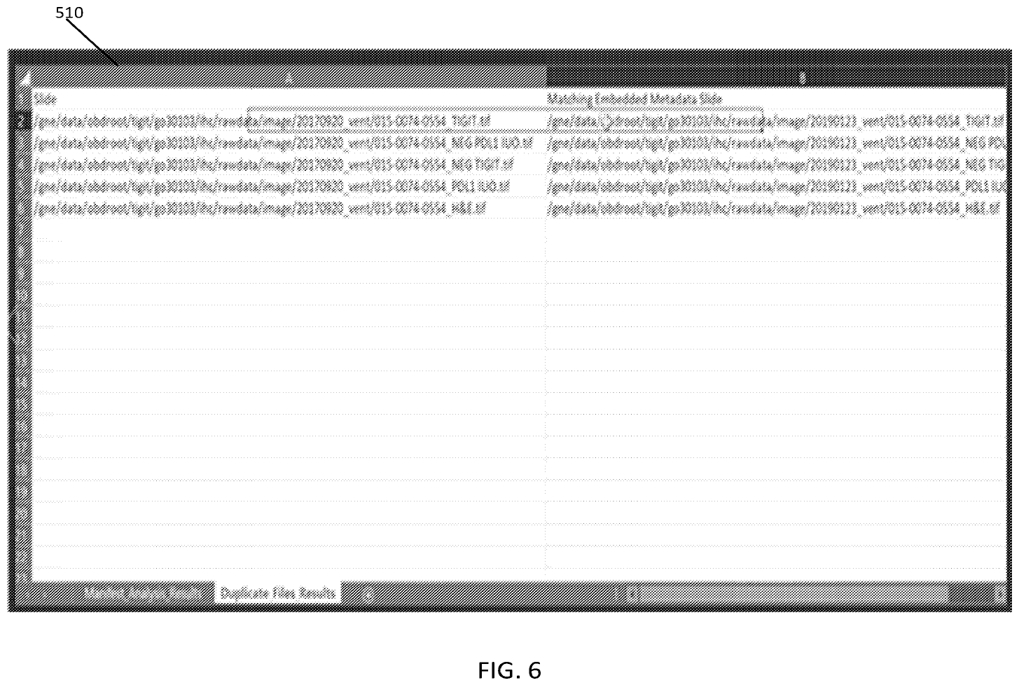

The database 120 may store identification information 125 associated with the image set 115. The identification information 125 may include a manifest 510, an example of which is shown in FIGS. 5 and 6. The manifest 510 may include a directory for the image set 115 including the information associated with the image set 115. The information stored in the manifest 510 may include one or more identifiers, such as one or more alpha, numeric, alphanumeric, or the like, identifiers. The identifiers may include, for each image, a slide identifier, a tissue type or tissue identifier, a specimen or sample identifier 514, a block identifier 512, a patient identifier, or the like. Referring to FIG. 5, each row of the manifest 510 corresponds to a particular slide image and each column of the manifest 510 corresponds to the information, such as the various identifiers.

Referring again to FIG. 1, as noted, the deduplication engine 110 may identify one or more duplicates, replicates, and/or multiples present within the image set 115. In some instances, the image set 115 may be selected, from amongst other image sets stored in the database 120, as an input for the deduplication engine 110. In some implementations, the database 120 may store the image set 115 and the corresponding identification information 125 in various formats and/or uses differing naming conventions. Thus, the deduplication engine 110 may standardize the identification information 125 associated with the image set 115 such that the identification information 125 can be compared by the deduplication engine 110. The deduplication engine 110 may standardize the identification information 125 before, during, or after the identification or removal of the replicates, duplicates, and/or multiples from the image set 115.

For example, the deduplication engine 110 may use autology techniques to standardize the identification information 125 associated with the image set 115. Autology techniques may include tools for curating large metadata sets, such as the identification information 125 associated with the image set 115, while arbitrating discrepancies within the identification information 125. Using the autology techniques described herein, the deduplication engine 110 may accelerate and/or improve curation of the identification information 125. For example, two images of a same tissue sample originating from a same organ may refer to the organ by different names. Accordingly, some images of the same tissue samples may include identifiers having different naming conventions. The deduplication engine 110 may standardize the identification information 125, such as the identifiers contained therein, to enable a subsequent comparison of the identification information 125.

In some implementations, the autology techniques used by the deduplication engine 110 may include using a Levenshtein distance to find similar terms that correspond to a same tissue sample, such as a same tissue sample identifier. The Levenshtein distance is a metric that measures a distance between two sequences of words. For example, the Levenshtein distance may include a Levenshtein ratio, partial ratio, token sort ratio, token set ratio, or the like. The Levenshtein ratio, partial ratio, token set ratio, and/or token sort ratio indicates a similarity between the two sequences, such as two identifiers of the information associated with the image set 115. The deduplication engine 110 may standardize the identification information 125 associated with the image set 115 by, for example, adjusting two identifiers to match when the two identifiers have a Levenshtein distance that meets a threshold. Standardizing the identification information 125 associated with the image set 115 can help improve performance of the deduplication engine 110 in identifying replicates, duplicates, and/or multiples within the image set 115.

Referring again to FIG. 1, the deduplication engine 110 may compare the identification information 125 associated with the image set 115 (e.g., a plurality of images of a plurality of tissue samples) to determine whether two or images (e.g., a first image and a second image) are replicates, duplicates, and/or multiples. The identification information 125 may include the manifest 510 as well as metadata 520, which may be embedded with the image set 115 and/or the individual images contained therein. In some cases, the metadata 520 may include a first timestamp at which the first image was acquired, a second timestamp at which the second image was acquired, a first modality used to acquire the first image, a second modality used to acquire the second image, a first slide identifier associated with the first tissue sample, a second slide identifier associated with the second tissue sample, a first barcode associated with the first tissue sample, a second barcode associated with the second tissue sample, a first scanning parameter associated with the first image, and a second scanning parameter associated with the second image.

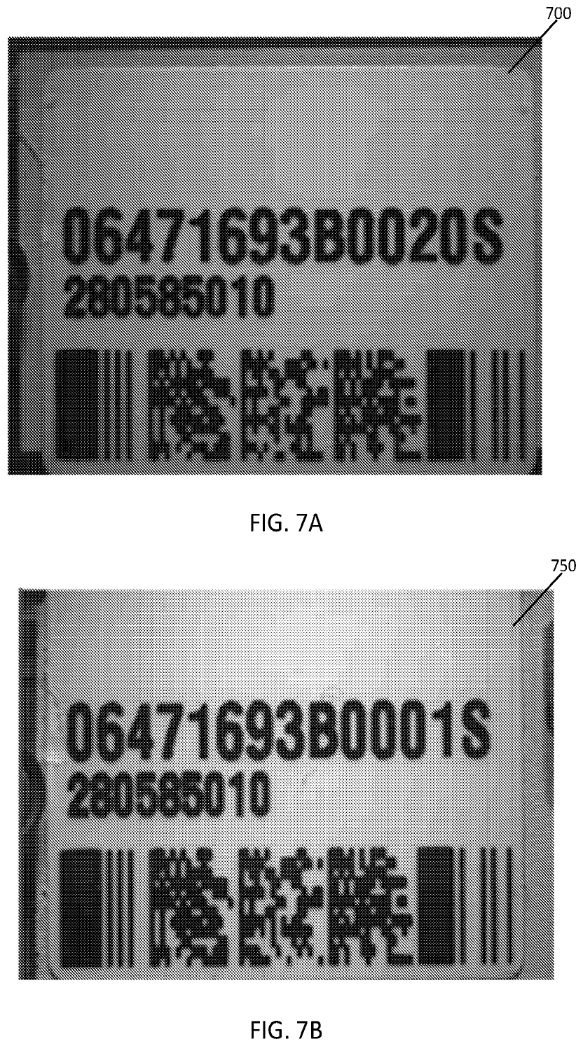

In some cases, where the metadata 520 of the image set 115 includes the barcode associated with each corresponding tissue sample, the barcode may encode at least a portion of the information associated with each individual image and/or the tissue sample depicted therein. Alternatively and/or additionally, at least a portion of the identification information 125 associated with the image and/or the tissue sample depicted in the image may be retrieve, for example, from the database 120, based on a corresponding resource identifier (e.g., a uniform resource locator (URL) of the database 120). FIG. 7A depicts a first example of a barcode 700 associated with the first tissue sample depicted in the first image while FIG. 7B depicts a second example of a barcode 750 associated with the second tissue sample depicted in the second image. While the barcode 700 and the barcode 750 are shown as two dimensional matrix barcodes (e.g., quick response (QR) codes), the barcode associated with each tissue sample may also be implemented as linear barcodes as well as electromagnetic tags such as radio frequency identification (RFID) tags and near field communication (NFC) tags.

To compare the identification information 125, the deduplication engine 110 may be implemented as a tool, such as an automated tool. The tool may be run as a command line tool running on Python 3, among other systems. As an example, the client device 130 may receive an input including a selection of a sample identifier, a patient identifier, or the like. The input may additionally or alternatively include a file format of the image set 115, a name of the sample identifier 514 in the manifest 510, a name of the block identifier 512 in the manifest 510, an input directory or directories including the image set 115 and corresponding manifest 510, an output directory for saving the results, or the like. The tool may read image files and/or manifests, such as the manifest 510.

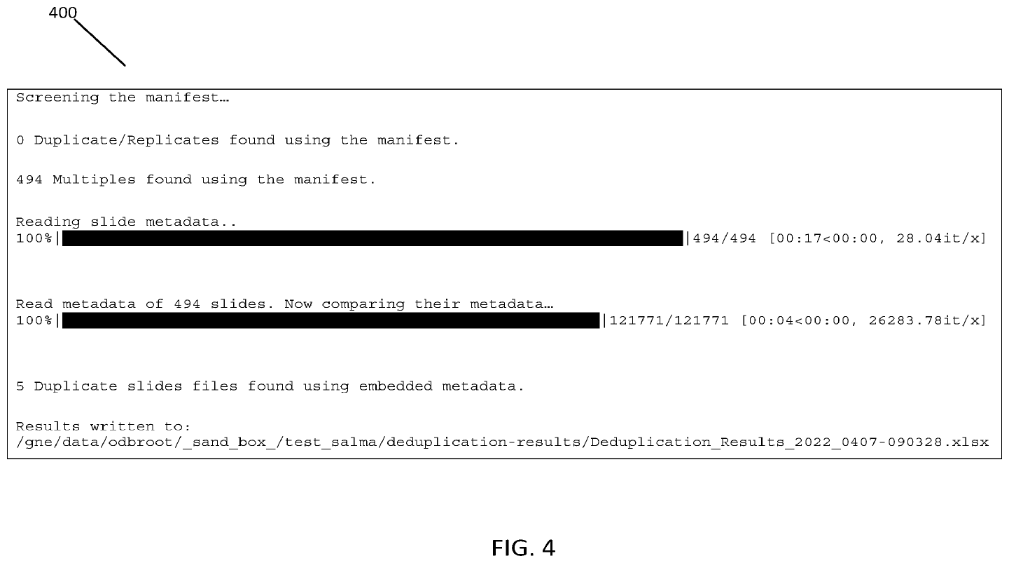

FIG. 4 shows an example of an implementation 400 of the tool including an output of the tool. Based on the comparison of the identification information 125, the deduplication engine 110 may identify at least one or more duplicate images, replicate images, and/or multiple images present within the image set 115.

In response to the first image being identified as a multiple, replicate, and/or duplicate of the second image based on the comparison of the identification information 125 associated with each of the first image and the second image, the deduplication engine 110 may update the identification information 125 to indicate the first image as a multiple, replicate, and/or duplicate (e.g., by including one or more corresponding flags in the manifest 510). In cases where the first image is determined to be a duplicate of the second image (e.g., depicting a same slice of tissue), the first image may be removed from the image set 115. It should be appreciated that the deduplication engine 110 may continue to deduplicate, based on the identification information 125, the remaining images in the image set 115 and make corresponding updates to the identification information 125. Moreover, in some example embodiments, the deduplication engine 110 may also deduplicate the image set 115 based on a metric indicative of a minimal difference achieved between the each pair of images in the image set 115 while adjusting an alignment therebetween. In some cases, the metric-based deduplication may be performed in order to identify duplicate images, replicate images, and/or multiple images that the deduplication engine 110 failed to detect based on the identification information 125. Alternatively and/or additionally, the deduplication engine 110 may perform metric-based deduplication to correct any errors and/or discrepancies that may be present within the identification information 125 such as a mislabeling of duplicate images depicting the same slice of tissue, replicate images depicting adjacent slices of tissue from the same block of a tissue sample, and/or multiple images depicting slices of tissue from adjacent blocks of the tissue sample.

In some example embodiments, the deduplication engine 110 may generate the metric based on a comparison of the vector fields present in one or more pairs of images in the image set 125. Doing so may prevent the deduplication and ordering of the images from being compromised due to misalignments between the images. Accordingly, the comparison of the vector fields present in each pair of images may be performed while aligning the images to achieve a minimal difference in the vector fields therebetween. Alignment in this case may include identifying the center of each image and aligning the images based on the center of the images. Once the images are centered, alignment may further include reorienting the images to correct for any inadvertent rotations (e.g., clockwise or counter clockwise) and inversions of the images, which may occur during the scanning of the images.

Where the difference between the vector fields of two aligned images satisfy a first threshold (e.g., a first value x that is near zero), the deduplication engine 110 may determine that the two images are duplicates of one another. Alternatively, where the difference between the vector fields of the two images fails to satisfy the first threshold but satisfies a second threshold that is greater than the first threshold (e.g., a second value y that is greater than the first value x), the deduplication engine 110 may determine that the two images are replicates and/or multiples. In some cases, where the difference between the vector fields of the two images satisfies the second threshold, the deduplication engine 110 may determine that the two images are images of adjacent slices of tissue from the same block of the tissue sample or images from two adjacent blocks of the tissue sample.

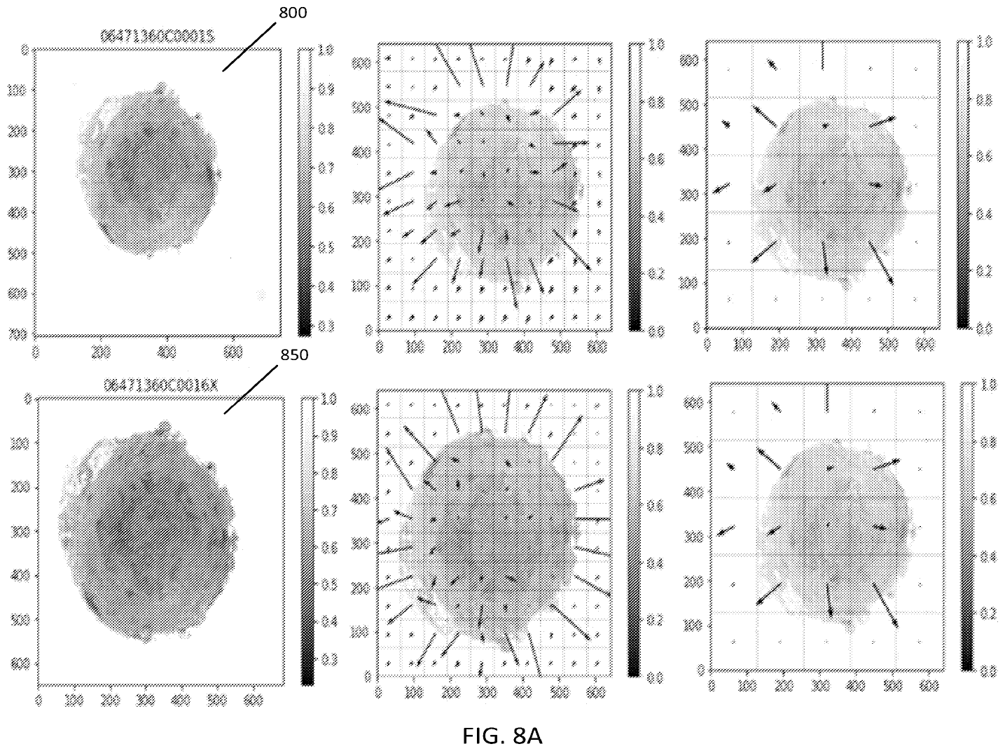

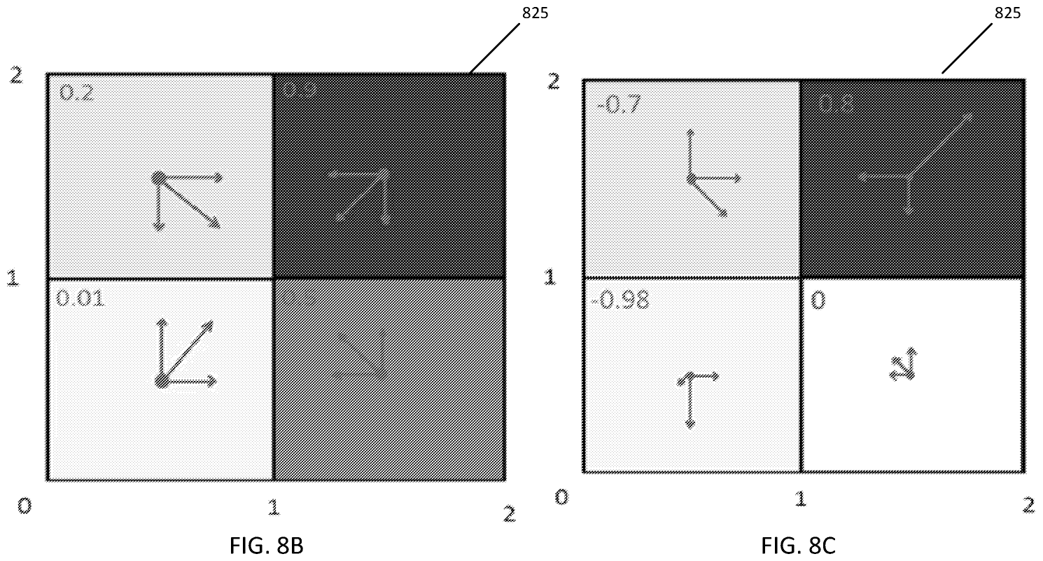

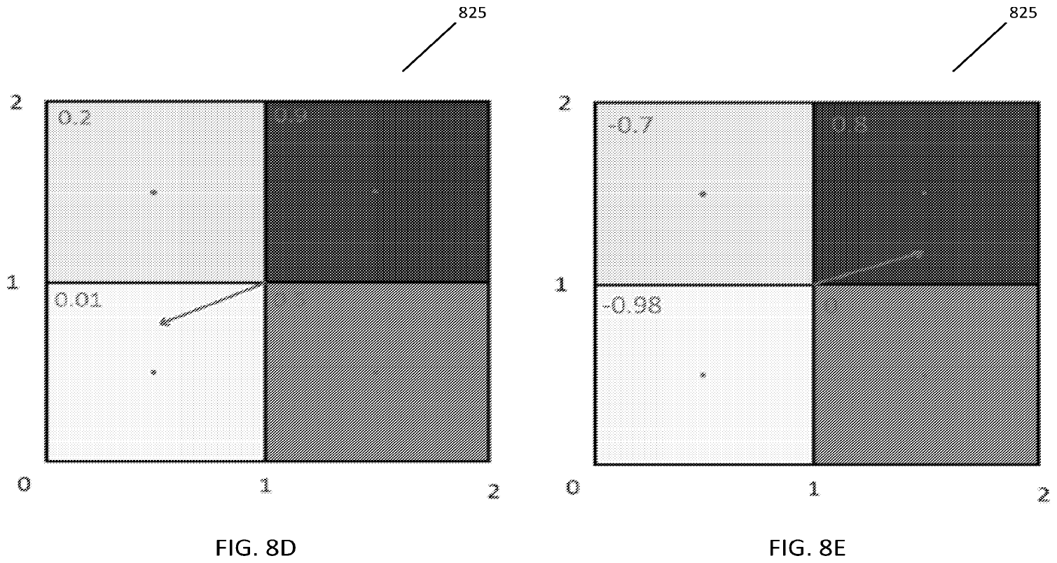

As used herein, the vector field of an image may include one or more vectors, each of which corresponding to a section of the image and having a direction and a magnitude (e.g. length) corresponding to a change in the intensity values of the pixels (e.g., from black to white) included in the sections of the image. To further illustrate, FIG. 8A depicts examples of vector fields found in a first image 800 and a second image 850, which are two similar and contiguous images from the same block of a tissue sample. To determine the respective vector fields of the first image 800 and the second image 850, the deduplication engine 110 may divide each image into equal sized sections such as in a grid. FIG. 8A shows the first image 800 and the second image 850 being divided into different sized grids such as, for example, grids in which each section contains 8 pixels, 16 pixels, 32 pixels, 64 pixels, or 128 pixels. Moreover, the deduplication engine 110 may convert the first image 800 and the second image 850 into grayscale images before changing the scale of the pixel intensity values from a first scale [0,1] to a second scale [−1,1]. Accordingly, the vector field for each of the first image 800 and the second image 850 may include a vector for each section of the image. The direction and magnitude (e.g., length) of those vectors may be representative of the change in the intensity values of the pixels (e.g., from black to white) included in the corresponding sections of the images.

In some example embodiments, the deduplication engine 110 may convert the scale of pixel intensity from the first scale [0,1] to the second scale [−1,1]. In cases where the first image 800 and the second image 850 are color images, the deduplication engine 110 may choose the first scale [0,1] or the second scale [−1,1] for each of the red, green, and/or blue components of the images. Nevertheless, it should be appreciated that either the first scale [0,1] or the second scale [−1,1] may be used for determining the vector fields of each of the first image 800 and the second image 850.

Although either the first scale [0,1] or the second scale [−1,1] may be used, converting the scale of pixel intensity from the first scale [0,1] to the second scale [−1,1] may change the directionality of the resulting vector fields. To further illustrate, FIGS. 8B-C depict different examples of a quadrant 825 containing four adjacent pixels from the first image 800. As shown in FIGS. 8B-C, each pixel in the quadrant 825 may be associated with one or more vectors originating from the center of the pixel. The deduplication engine 110 may determine a pixel gradient for the quadrant 825 corresponding to the vectors associated with each constituent pixel. For example, the deduplication engine 110 may determine the pixel gradient for the quadrant 825 by translating the vectors associated with each constituent pixel to the center of the quadrant 825 and computing a sum of the vectors.

FIGS. 8D-E depict examples of vector fields computed based on different scales for pixel intensity. For example, FIG. 8D depicts one example in which the vector field of the quadrant 825 is computed based on the first scale [0,1] while FIG. 8E depicts another example in which the vector field of the quadrant 825 is computed based on the second scale [−1,1]. As noted, either the first scale [0,1] or the second scale [−1,1] may be used but using one scale over the other may change the directionality of the resulting vector field. For instance, the example of the vector field shown in FIG. 8D, which is computed based on the first scale [0,1], may be point from the darker (or higher intensity) pixels in the quadrant 825 towards the lighter (or lower intensity) pixels in the quadrant 825. Contrastingly, the example of the vector field shown in FIG. 8E, which is computed based on the second scale [−1,1], may point from the lighter (or lower intensity) pixels in the quadrant 825 towards the darker (or higher intensity) pixels in the quadrant 825.

In some example embodiments, the pixel gradient for the quadrant 825 may be calculated separately for horizontal components and vertical components of the vectors associated with each constituent pixels in the quadrant 825. For example, in some cases, the pixel gradient Ex along the x-axis of the quadrant 825 may be determined by applying Equation (1) below whereas the pixel gradient E_{y} along the y-axis of the quadrant 825 may be determined by applying Equation (2).

$${E}_{x} = \frac{q \times dx}{1} \qquad ( 1 )$$

$${E}_{y} = \frac{q \times dy}{1} \qquad ( 2 )$$

wherein q denotes the intensity value of each pixel, dx denotes a change in distance along the x axis, and dy denotes a change in distance along the y axis. It should be appreciated that the values of dx and dy may be positive or negative depending on where the pixel is located relative to the center of the quadrant 825.

The magnitude of the vector at the center of each portion of the first image 800, such as the quadrant 825, may be determined by applying Equation (3) below to the individual x and y components.

$$E = \sqrt{{E}_{x}^{2} + {E}_{y}^{2}} \qquad ( 3 )$$

As noted, converting the intensity values of the pixels in the first image 800 from the first scale [0,1] to the second scale [−1,1] may change the directionality of the resulting vector fields, for example, from pointing in the direction of the lighter (or lower intensity) pixels to pointing in the direction of the darker (or higher intensity) pixels in the first image 800. This phenomenon is shown in FIGS. 8D-E. Nevertheless, either the first scale [0,1] or the second scale [−1,1] may be used to compare the vector fields of two different images, such as those of the first image 800 and the second image 850 shown in FIG. 8A, to determine whether two images are duplicate, replicates, or multiples of one another.

In some example embodiments, the deduplication engine 110 may determine, based on a first vector field of the first image 800 and a second vector field of the second image 850, whether the first image 800 and the second image 850 are multiples, replicates, and/or duplicates of one another. However, in some instances, a misalignment between the first image 800 and the second image 850 may prevent the deduplication engine 110 from identifying the first image 800 and the second image 850 as multiples, replicates, and/or duplicates of one another at least because any similarities in the respective vector fields would be obscured by a corresponding misalignment between the two vector fields. The misalignments between the first image 800 and the second image 850 may arise from translations, rotations, and/or inversions of one or both images during scanning. For example, the first image 800 and the second image 850 may be duplicate images but if the first image 800 is scanned upside down while the second image 850 is scanned right side up, the correspondence between the two images may be obscured by the inversion of the first image 800.

Accordingly, the deduplication engine 110 may determine the difference between the first vector field of the first image 800 and the second vector field of the second image 850 all while aligning the first image 800 and the second image 850 to minimize the difference between the first vector field and the second vector field. That is, the deduplication engine 110 may continue to adjust the alignment between the first image 800 and the second image 850 in order to minimize any possible misalignment between the two images when computing the difference between the corresponding vector fields. In doing so, the deduplication engine 110 is able to detect instances where the similarities between the first image 800 and the second image 850 are obscured by a misalignment between the first image 800 and the second 850. In some cases, while the deduplication engine 110 adjusts the alignment between the first image 800 and the second image 850, the deduplication engine 110 may determine the difference between the first vector field of the first image 800 and the second vector field of the second image 850 by at least subtracting the x-components of the first vector field and the second vector field separately from the y-components of the two vector fields.

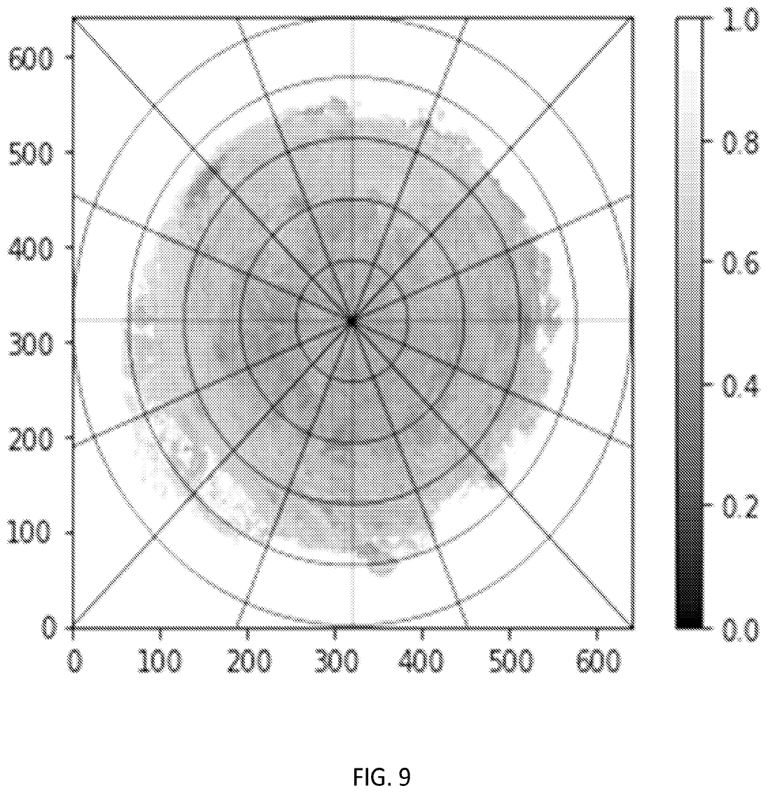

While determining the difference between the first vector field and the second vector field, the deduplication engine 110 may translate the two vector fields, for example, along the x-axis and/or the y-axis at grid-size intervals to determine an alignment of the first image 800 and the second image 850 where the difference between the two vector fields is minimized. Doing so may enable the deduplication engine 110 to detect instances where the similarities in the vector fields of the first image 800 and the second image 850 are obscured by a translational misalignment between the first image 800 and the second image 850. Alternatively and/or additionally, while determining the difference between the first vector field and the second vector field, the two vector fields may also be rotated and/or inverted to determine an alignment of the first image 800 and the second image 850 that minimizes the difference between the two vector fields. Rotating the two vector fields may enable the deduplication engine 110 to detect instances where the similarities in the vector fields of the first image 800 and the second image 850 are obscured by a rotational misalignment between the two images. Meanwhile, inverting one vector field relative to the other vector field may enable the deduplication engine 110 to detect instances where the similarities in the vector fields of the first image 800 and the second image 850 are obscured by an inversion of one of the images. To enable the rotation of a vector field about its center axis, the deduplication engine 110 may use polar coordinates such that the rotation of the vector fields is quantified in degree intervals. An example of a vector field mapped to polar coordinates is shown in FIG. 9. Meanwhile, to invert a vector field (e.g., rotate the vector field 180 degrees along its x-axis and y-axis), the deduplication engine 110 may apply a rotation matrix or quaternions. In some cases, in addition to translating, rotating, and/or inverting the first vector field and the second vector field, the deduplication engine 110 may also change the size of the sections in each of the first image 800 and the second image 850 (e.g., grid size) in order to identify a size (e.g., grid size) that is associated with a minimal difference in the vector fields.

In some example embodiments, the deduplication engine 110 may determine, based at least on the minimal difference between the first vector field of the first image 800 and the second vector field of the second image 850, whether the first image 800 and the second image 850 are multiples, replicates, and/or duplicates of one another. For example, where the difference between the first vector field of the first image 800 and the second vector field of the second image 850 satisfy the first threshold (e.g., a first value x that is near zero), the deduplication engine 110 may determine that the first image 800 and the second image 850 are duplicates of one another. Alternatively, where the difference between the first vector field of the first image 800 and the second vector field of the second image 850 fails to satisfy the first threshold but satisfies a second threshold that is greater than the first threshold (e.g., a second value y that is greater than the first value x), the deduplication engine 110 may determine that the first image 800 and the second image 850 replicates and/or multiples. In some cases, the first image 800 and the second image 850 may be identified as images of adjacent slices from the same block of the tissue sample when where the difference between their respective vector fields satisfies the second threshold. Identifying the first image 800 and the second image 850 as adjacent images may enable the deduplication engine 110 to restore the original sequence of the images from the block of the tissue sample.

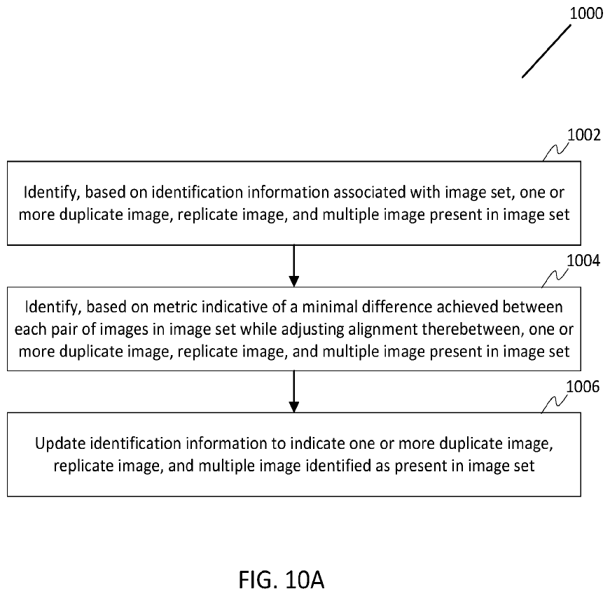

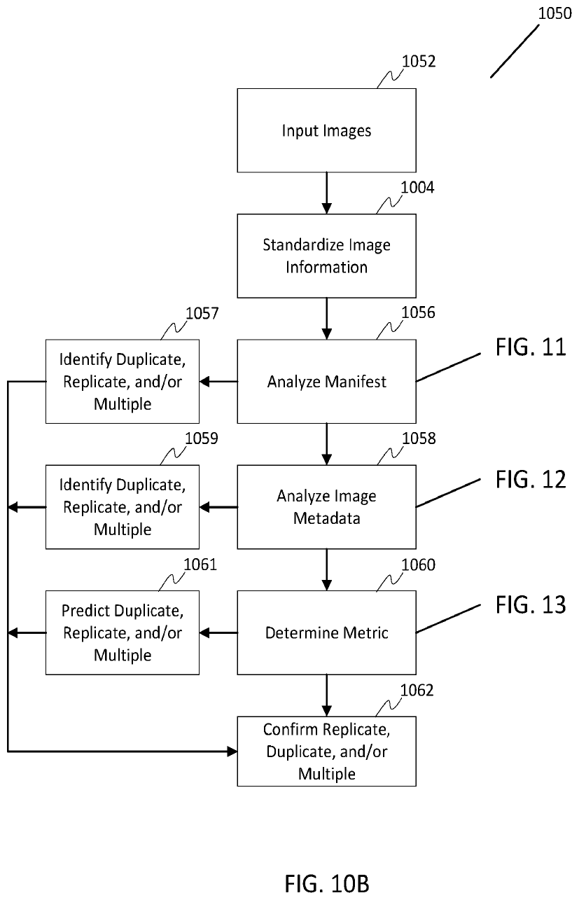

FIG. 10A depicts a flowchart illustrating an example of a process 1000 for identifying sample images, consistent with implementations of the current subject matter. Referring to FIGS. 1 and 10A, the process 1000 may be performed by the deduplication engine 110 to identify, within the image set 115, one or more duplicate images, replicate images, and/or multiple images. In some cases, the deduplication engine 110 may further perform the process 1000 to identify adjacent images in order to restore the images in the image set 115 to their original sequence in the block of the tissue sample from which the images originate.

At 1002, the deduplication engine 110 may identify, based on the identification information 125 associated with the image set 115, one or more of a duplicate image, a replicate image, and a multiple image present in the image set 115. In some example embodiments, the deduplication engine 110 may deduplicate the image set 115 based on the identification information 125 associated with the image set 115. The identification information 125 associated with the image set 115 may include the manifest 510 and the metadata 520, which may be stored as a part of the manifest 510 and/or embedded with the individual images in the image set 115. Moreover, the identification information 125 associated with the image set 115 may include a variety of data including, for example, a sample identifier, a patient identifier, a block identifier, a slide identifier, an imaging modality, a scanning parameter.

Accordingly, the deduplication engine 110 may identify a first image in the image set 115 as a duplicate of a second image in the image set 115 if the first image and the second image are associated with matching sample identifiers, patient identifiers, block identifiers, and slide identifiers. Where the first image and the second image are associated with matching sample identifiers, patient identifiers, and block identifiers but different slide identifiers, the deduplication engine 110 may identify the first image as a replicate of the second image. Furthermore, where the first image and the second image are associated with matching sample identifiers and patient identifiers but different block identifiers and different slide identifiers, the deduplication engine may identify the first image and the second image as multiples of one another.

At 1004, the deduplication engine 110 may identify, based at least on a metric indicative of a minimal difference achieved between each pair of images in the image set 115 while adjusting an alignment therebetween, the one or more of a duplicate image, a replicate image, and a multiple image present in the image set 115. In some example embodiments, the deduplication engine 110 may deduplicate the image set 115 based on a metric indicative of a minimal difference achieved between each pair of images in the image set 115, such as a minimal difference in the respective vector fields of the two images, while adjusting the alignment therebetween. For example, the deduplication engine 110 may determine the difference between the first vector field of the first image 800 and the second vector field of the second image 850 while adjusting the alignment between the first image 800 and the second image 850 through translation, rotation, inversion, and/or the like. The minimal difference that is achieved between the two vector fields while adjusting the alignment between the first image 800 and the second image 850 may be indicative of the similarities between the two images or lack thereof. For instance, where the difference between the first vector field of the first image 800 and the second vector field of the second image 850 satisfy the first threshold (e.g., a first value x that is near zero), the deduplication engine 110 may determine that the first image 800 and the second image 850 are duplicates of one another. Alternatively and/or additionally, where the difference between their respective vector fields does not satisfy the first threshold but satisfies a second threshold that is greater than the first threshold, the first image 800 and the second image 850 may be identified as replicates, and more specifically, adjacent images from the same block of the tissue sample.

In some cases, the metric-based deduplication may be performed on the remaining images in the image set 115 subsequent to the deduplication performed based on the identification information 125. That is, once the deduplication engine 110 identified one or more duplicate images, replicate images, and/or multiple images in the image set 115 based on the identification information 125, the deduplication engine 110 may perform metric-based deduplication on the remainder of the image set 115. Alternatively, after the deduplication engine 110 identified one or more duplicate images, replicate images, and/or multiple images in the image set 115 based on the identification information 125, the deduplication engine 110 may perform metric-based deduplication on the entirety of the image set 115. Doing so may enable the deduplication engine 110 to detect any errors that may be present in the identification information 125.

At 1006, the deduplication engine 110 may update the identification information 125 associated with the image set 115 to indicate the one or more of the duplicate image, the replicate image, and the multiple image identified as present within the image set 115. For instance, in some example embodiments, the deduplication engine 110 may update the identification information 125 (e.g., the manifest 510, the metadata 520, and/or the like) by at least setting one or more flags to indicate whether each image in the image set 115 is a duplicate, replicate, and/or multiple. In some cases, the deduplication engine 110 may also update the identification information 125 to indicate the sequence of the images within the image set 115. For example, the identification information 125 may be updated to identify replicate images depicting adjacent slices of tissue from the same block of the tissue sample and multiple images depicting slices of tissue from adjacent blocks of the tissue sample. Alternatively and/or additionally, the deduplication engine 110 may update the identification information 125 to correct for any discrepancies between the identification information 125 and the one or more deduplicate images, replicate images, and multiple images identified by the deduplication engine 110 through metric-based deduplication.

FIG. 10B depicts a flowchart illustrating an example of a process 1050 for identifying sample images, consistent with implementations of the current subject matter. Referring to FIGS. 1 and 10B, the process 1050 may be performed by the deduplication engine 110 to identify, within the image set 115, one or more duplicate images, replicate images, and/or multiple images. In some cases, the deduplication engine 110 may further perform the process 1050 to identify adjacent images in order to restore the images in the image set 115 to their original sequence in the block of the tissue sample from which the images originate.

At 1052, the deduplication engine 110 may receive the image set 115. For example, the deduplication engine 110 may receive, from the database 120, the image set 115, which may be a set of whole slide images depicting slices of tissue from one or more blocks of a tissue sample of a patient. In some cases, the image set 115 may include duplicate images, which are identical or near-identical whole slide images depicting the same tissue slice. Alternatively and/or additionally, the image set 115 may include replicate images, which are whole slide images depicting adjacent tissue slices from the same block of the tissue sample. In some instances, the image set 115 may also include multiple images, which are whole slide images depicting tissue slices from different blocks of the tissue specimen. In the case of replicates and multiples, the image set 115 may include whole slide images depicting adjacent slices of the tissue sample as well as whole slide images depicting slices of the tissue sample from adjacent blocks of the tissue sample. Replicate images and/or multiple images in the image set 115 may, in some cases, be out of their original sequence within individual blocks and across adjacent blocks.

At 1054, the deduplication engine 110 may standardize the identification information 125 associated with the image set 115. In some example embodiments, the deduplication engine 110 may standardize the identification information 125 associated with the image set 115 by at least reconciling the different formats and naming conventions that may be present therein. To do so, the deduplication engine 110 may apply one or more techniques, including autology techniques such as Levenshtein distance, to standardize the identification information 125 associated with the image set 115. The standardization of the identification information 125 may enable the deduplication engine 110 to perform a subsequent analysis of the identification information 125.

At 1056, the deduplication engine 110 analyze the manifest 510 associated with the image set 115. At 1057, the deduplication engine 110 may identify, based on the analysis of the manifest 510, one or more duplicate, replicate, and/or multiple images present in the image set 115. For example, the manifest 510 associated with the image set 115 may include one or more identifiers, such as one or more alpha, numeric, alphanumeric, or the like, identifiers. Furthermore, the manifest 510 may include, for each image included in the image set 115, a slide identifier, a tissue type or tissue identifier, a specimen or sample identifier, a block identifier, a patient identifier, and/or the like. Accordingly, the deduplication engine 110 may analyze the manifest 510 associated with the image set 115 to detect, for example, identical and/or sequential identifiers indicative of one or more duplicate, replicate, and/or multiple images present within the image set 115.

At 1058, the deduplication engine 110 may analyze the metadata 520 associated with the image set 115. At 1059, the deduplication engine 110 may identify, based on the analysis of the metadata 520, one or more duplicate, replicate, and/or multiple images present in the image set 115. The metadata 520 associated with the image set 520 may be stored as a part of the manifest 510 and/or embedded within the individual images in the image set 115. In some cases, the metadata 520 may include image timestamps, image modalities, slide identifiers, barcodes, scanning parameters, and/or the like. Accordingly, in some example embodiments, the deduplication engine 110 may also analyze the metadata 520 associated with the image set 115 detect one or more duplicate, replicate, and/or multiple images present within the image set 115.

At 1060, the deduplication engine 110 may determine a metric associated with the image set 115. At 1061, the deduplication engine 110 may predict, based on the metric, one or more one or more duplicate, replicate, and/or multiple images present in the image set 115. In some example embodiments, the deduplication engine 110 may determine a metric corresponding to a difference in the vector fields between one or more pairs of images in the image set 115. In some cases, the deduplication engine 110 may align each pair of images in the image set 115 such that the difference in the respective vector fields of the images is minimized. The presence of duplicates, replicates, and/or multiples may be determined based on the minimal difference in the vector fields achieved by adjusting the alignment between each pair of images. For example, where the difference between the vector fields of two aligned images satisfy a first threshold (e.g., a first value x that is near zero), the deduplication engine 110 may determine that the two images are duplicates of one another. Alternatively, where the difference between the vector fields of the two images fails to satisfy the first threshold but satisfies a second threshold that is greater than the first threshold (e.g., a second value y that is greater than the first value x), the deduplication engine 110 may determine that the two images are replicates and/or multiples. In some cases, where the difference between the vector fields of the two images satisfy the second threshold but not the first threshold, the deduplication engine 110 may determine that the two images depict adjacent tissue slices from the same block of the tissue sample.

At 1062, the deduplication engine 110 may confirm the one or more images identified as a replicate, duplicate, and/or a multiple. For example, in some example embodiments, the deduplication engine 110 may confirm the one or more images identified as a duplicate before flagging and/or removing the one or more images from the image set 115. In some cases, the deduplication engine 110 may also confirm, based on one or more user inputs, the one or more images identified as depicting adjacent slices from the same block of the tissue sample before restoring the original sequence of the images from the block of the tissue sample. For instance, the one or more user inputs may indicate whether the one or more images are correctly identified as depicting adjacent slices from the same block of the tissue sample, in which case the deduplication engine 110 may adjust, based at least on the ordering of the one or more images identified as depicting adjacent slices from the same block of the tissue sample, the current sequence of images to restore the original sequence of the images. Where the one or more user inputs rejects the one or more images identified as depicting adjacent slices from the same block of the tissue sample, the deduplication engine 110 may maintain the current sequence of images.

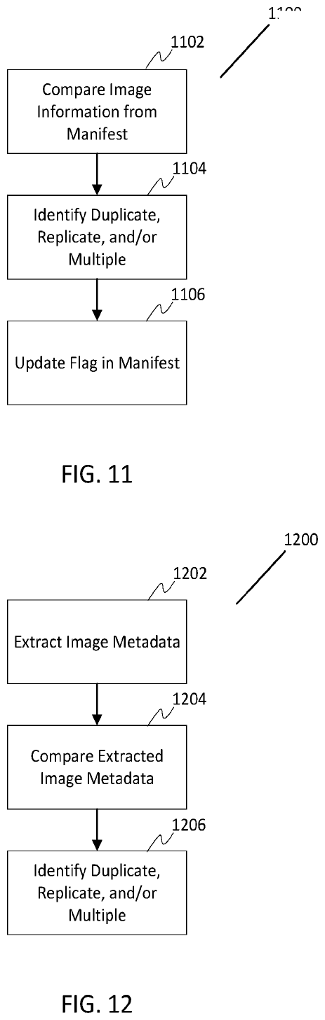

FIG. 11 depicts a flowchart illustrating an example of a process 1100 for identifying duplicate, replicate, and/or multiple images, consistent with implementations of the current subject matter. Referring to FIGS. 1, 10A-B, and 11, the process 1100 may be performed by the deduplication engine 110 to identify one or more multiple, replicate, and/or duplicate images within the image set 115. In some example embodiments, the process 1100 may implement operation 1002 of the process 100 shown in FIG. 10A and operation 1056 of the process 1050 shown in FIG. 10B.

At 1102, the deduplication engine 110 may compare image information from the manifest 400 associated with a set of input images. At 1104, the deduplication engine 110 may identify, based on the comparison of image information from the manifest 510, one or more duplicates, replicates, and/or multiples present in the image set 115. As noted, the manifest 510 for the image set 115 may include one or more identifiers including, for example, alpha, numeric, and/or alphanumeric identifiers. Examples of such identifiers may include, for each image included in the image set 115, a slide identifier, a tissue type or tissue identifier, a specimen or sample identifier, a block identifier, a patient identifier, and/or the like. Accordingly, the deduplication engine 110 may analyze the manifest 510 of the image set 115 to identify one or more duplicates, replicates, and/or multiples.

At 1106, the deduplication engine 110 may update the manifest 510 to indicate the one or more duplicates, replicates, and/or multiples identified as present in the image set 115. In some example embodiments, the manifest 115 of the image set 115 may include one or more columns occupied by one or more flags identifying the corresponding images as a replicate, a duplicate, and/or a multiple. Accordingly, the deduplication engine 110 may, based on whether an image is identified as a duplicate, a replicate, and/or a multiple, one or more corresponding flags in the manifest 510 of the image set 115.

FIG. 12 depicts a flowchart illustrating another example of a process 1200 for identifying images, consistent with implementations of the current subject matter. Referring to FIGS. 1, 10A-B, and 12, the process 1200 may be performed by the deduplication engine 110 to identify one or more duplicate images within the image set 115. In some example embodiments, the process 1100 may implement operation 1002 of the process 1000 shown in FIG. 10A and operation 1058 of the process 1050 shown in FIG. 10B.

At 1202, the deduplication engine 110 may extract the metadata 520 associated with the image set 115. At 1204, the deduplication engine 110 may compare the extracted metadata 520. In some example embodiments, the metadata 520 associated with image set 115 may be stored as a part of the manifest 510 and/or embedded with the individual images in the image set 115. As noted, in some cases, the metadata 520 may include image timestamps, image modalities, slide identifiers, barcodes, scanning parameters, and/or the like. Accordingly, the deduplication engine 110 may compare the metadata 520 associated with a first image and a second image by at least comparing, for example, a first timestamp at which the first image was acquired, a second timestamp at which the second image was acquired, a first modality used to acquire the first image, a second modality used to acquire the second image, a first slide identifier associated with the first tissue sample, a second slide identifier associated with the second tissue sample, a first barcode associated with the first tissue sample, a second barcode associated with the second tissue sample, a first scanning parameter associated with the first image, and a second scanning parameter associated with the second image.

At 1206, the deduplication engine 110 may identify, based at least on the comparison of the extracted metadata 520, one or more duplicates, replicates, and/or multiples. In some example embodiments, one or more duplicates, replicates, and/or multiples present within the image set 115 may be identified based on the comparison of the corresponding metadata 520. Moreover, in some cases, the deduplication engine 110 may update the manifest 510 of the image set 115, for example, by setting one or more corresponding flags, to indicate which images are duplicates, replicates, and/or multiples.

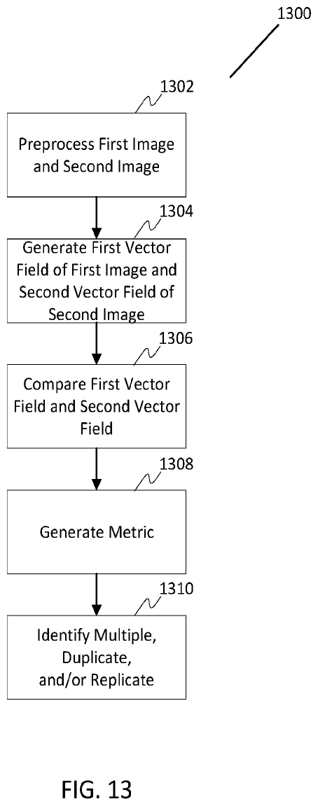

FIG. 13 depicts a flowchart illustrating another example of a process for predicting duplicate and/or replicate images, consistent with implementations of the current subject matter. Referring to FIGS. 1, 10A-B, and 13, the process 1300 may be performed by the deduplication engine 110 to identify one or more duplicate and/or replicate images within the image set 115. In some cases, the deduplication engine 110 may further perform the process 1300 to identify adjacent images in order to restore the images in the image set 115 to their original sequence in the block of the tissue sample from which the images originate. Moreover, in some example embodiments, the process 1300 may implement operation 1004 of the process 1000 shown in FIG. 10A and operation 1058 of the process 1050 shown in FIG. 10B.

At 1302, the deduplication engine 110 may preprocess a first image and a second image. In some example embodiments, the deduplication engine 110 may preprocess the first image and the second image by converting the images into grayscale images. Furthermore, the deduplication engine 110 may preprocess the first image and the second image by dividing each image into equal sized sections (e.g., a grid and/or the like). In some cases, the preprocessing of the first image and the second image may further include converting the scale of pixel intensity values from a first scale [0,1] to a second scale [−1,1].

At 1304, the deduplication engine 110 may generate a first vector field for the first image and a second vector field for the second image. In some example embodiments, the vector field for an image may include, for each section within the image, a vector whose direction and magnitude (e.g., length) correspond to the change in the intensity values of the pixels (e.g., from black to white) included in the section of the image.

At 1306, the deduplication engine 110 may compare the first vector field of the first image and the second vector field of the second image. For example, in some cases, the deduplication engine 110 may compare the first vector field of the first image and the second vector field of the second image by at least subtracting the x-components of the first vector field and the second vector field separately from the y-components of the two vector fields. In some example embodiments, as a part of comparing the first vector field of the first image and the second vector field of the second image, the deduplication engine 110 may also align the first image and the second image to minimize the difference between the two vector fields. For example, while determining the difference between the first vector field and the second vector field, the deduplication engine 110 may translate the two vector fields, for example, along the x-axis and/or the y-axis at grid-size intervals to determine an alignment of the two images where the difference between the two vector fields is minimized. Alternatively and/or additionally, while determining the difference between the first vector field and the second vector field, the two vector fields may also be rotated and/or inverted to determine an alignment of the first image and the second image that minimizes the difference between the two vector fields.

At 1308, the deduplication engine 110 may generate, based at least on the comparison of the first vector field and the second vector field, a metric. In some example embodiments, the deduplication engine 110 may generate a metric corresponding to the minimal difference in the first vector field and the second vector field that the deduplication engine 110 was able to achieve while adjusting the alignment between the first image and the second image.

At 1310, the deduplication engine 110 may identify, based at least on the metric, the first image and/or the second image as a replicate, a duplicate, and/or a multiple. In some example embodiments, the deduplication engine 110 may determine, based at least on the minimal difference between the first vector field of the first image and the second vector field of the second image, whether the first image and the second image are multiples, replicates, and/or duplicates of one another. For example, where the difference between the first vector field and the second vector field satisfy a first threshold (e.g., a first value x that is near zero), the deduplication engine 110 may determine that the two corresponding images are duplicates of one another. Alternatively, where the difference between the two vector fields fails to satisfy the first threshold but satisfies a second threshold that is greater than the first threshold (e.g., a second value y that is greater than the first value x), the deduplication engine 110 may determine that the first image and the second image are replicates and/or multiples.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:
Item 1: A computer-implemented method, comprising: identifying, based at least on an identification information associated with a plurality of images depicting one or more slices of tissue from at least one block of a tissue sample, one or more of a duplicate image, a replicate image, and a multiple image present within the plurality of images; identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and updating the identification information associated with the plurality of images to indicate the one or more of the duplicate image, the replicate image, and the multiple image identified as present within the plurality of images.
Item 2: The method of Item 1, wherein the identification information comprises at least a portion of a manifest, a scannable barcode, and/or metadata associated with the plurality of images.
Item 3: The method of any of Items 1 to 2, wherein the identification information includes one or more of a sample identifier, a patient identifier, a block identifier, a slide identifier, an imaging modality, and a scanning parameter.
Item 4: The method of Item 3, wherein a first image of the plurality of images is identified as a duplicate of a second image of the plurality of images based at least on the first image and the second image being associated with matching sample identifiers, patient identifiers, block identifiers, and slide identifiers.
Item 5: The method of any of Items 3 to 4, wherein a first image of the plurality of images is identified as a replicate of a second image of the plurality of images based at least on the first image and the second image being associated with matching sample identifiers, patient identifiers, and block identifiers but different slide identifiers.
Item 6: The method of any of Items 3 to 5, wherein a first image of the plurality of images is identified as a multiple of a second image of the plurality of images based at least on the first image and the second image being associated with matching sample identifiers and patient identifiers but different block identifiers and different slide identifiers.
Item 7: The method of any of Items 1 to 6, wherein the identification information is updated by at least including, in the identification information, a flag indicating the one or more of the duplicate image, the replicate image, and the multiple image present within the plurality of images.
Item 8: The method of any of Items 1 to 7, wherein the minimal difference between each pair of images corresponds to a minimal difference between a first vector field of a first image included in each pair of images and a second vector field of a second image included in each pair images.
Item 9: The method of Item 8, further comprising: generating the first vector field and the second vector field; and determining the difference between the first vector field and the second vector field while adjusting the alignment between the first image and the second image until achieving the minimal difference between the first vector field and the second vector field.
Item 10: The method of Item 9, wherein each of the first vector field and the second vector field comprises a plurality of vectors, wherein each vector of the plurality of vectors corresponds to a section within a corresponding image, and wherein each vector of the plurality of vectors has a direction and a magnitude corresponding to a change in an intensity values of one or more pixels included a corresponding section of the corresponding image.
Item 11: The method of Item 10, further comprising: dividing, into a plurality of sections, each of the first image and the second image; and adjusting a size of each section of the plurality of sections and/or a quantity of the plurality of sections until the minimal difference between the first vector field and the second vector field is achieved.
Item 12: The method of any of Items 9 to 11, wherein the alignment between the first image and the second image is adjusted by at least translating, rotating, and/or inverting the first image relative to the second image.
Item 13: The method of any of Items 9 to 12, wherein the difference between the first vector field and the second vector field is determined by at least subtracting one or more x-components of the first vector field and the second vector field separately from one or more y-components of the first vector field and the second vector field.
Item 14: The method of any of Items 1 to 13, wherein a first image in each pair of images is identified as a duplicate of a second image in each pair of images based at least on the metric satisfying a first threshold.
Item 15: The method of Item 14, wherein the first image is identified as a replicate of the second image based at least on the metric failing to satisfy the first threshold but satisfying a second threshold greater than the first threshold.
Item 16: The method of Item 15, further comprising: identifying, based at least on the metric satisfying the second threshold, the first image and the second image as adjacent images from a same block of the tissue sample; and updating the identification information associated with the plurality of images to indicate the first image and the second image as adjacent images from the same block of the tissue sample.
Item 17: The method of any of Items 9 to 16, further comprising: converting the first image and the second image into grayscale images prior to generating the first vector field and the second vector field.
Item 18: The method of any of Items 9 to 17, further comprising: converting, from a first scale to a second scale, a scale of pixel intensity values in each of the first image and the second image prior to generating the first vector field and the second vector field.
Item 19: The method of Item 18, wherein the first scale comprises [0, 1], and wherein the second scale comprises [−1, 1].
Item 20: The method of any of Items 1 to 19, wherein the one or more of the duplicate image, the replicate image, and the multiple image present within the plurality of images are first identified based on the identification information before the metric is computed for a remaining plurality of images to identify the one or more of the duplicate image, the replicate images, and the multiple images in the remaining plurality of images.
Item 21: The method of any of Items 1 to 20, further comprising: deleting at least one duplicate image present within the plurality of images.
Item 22: The method of any of Items 1 to 21, further comprising: updating the identification information to correct at least one discrepancy between the identification information and the one or more of the duplicate image, the replicate image, and the multiple image identified based on the metric.
Item 23: The method of any of Items 1 to 22, further comprising: updating a sequence of the plurality of images indicated in the identification information to restore, based at least on an ordering of at least one replicate image identified within the plurality of images, an original sequence of images within the at least one block of the tissue sample.
Item 24: The method of any of Items 1 to 23, further comprising: updating a sequence of the plurality of images indicated in the identification information to restore, based at least on an ordering of at least one multiple image identified within the plurality of images, an original sequence of images across different blocks of the tissue sample.
Item 25: The method of any of Items 1 to 24, wherein the duplicate image depicts a same slice of tissue as another image included in the plurality of images, wherein the replicate image depicts a different slice of tissue from a same block of the tissue sample as the another image included in the plurality of images, and wherein the multiple image depicts a slice of tissue from a different block of the tissue sample as the another image from the plurality of images.
Item 26: A system, comprising: at least one data processor; and at least one memory storing instructions, which when executed by the at least one data processor, result in operations comprising the method of any of Items 1 to 25.
Item 27: A non-transitory computer readable medium storing instructions, which when executed by at least one data processor, result in operations comprising the method of any of Items 1 to 25.

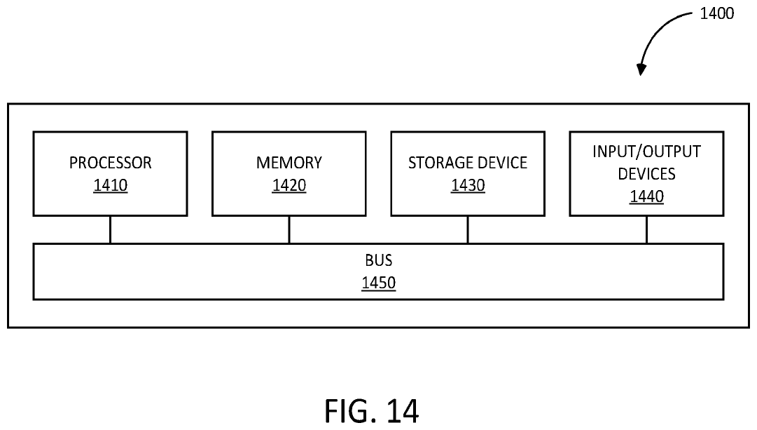

FIG. 14 depicts a block diagram illustrating a computing system 1400 consistent with implementations of the current subject matter. Referring to FIGS. 1-14, the computing system 1400 can be used to implement the deduplication engine 110, and/or any components therein.

As shown in FIG. 14, the computing system 1400 can include a processor 1410, a memory 1420, a storage device 1430, and input/output devices 1440. The processor 1410, the memory 1420, the storage device 1430, and the input/output devices 1440 can be interconnected via a system bus 1450. The computing system 1400 may additionally or alternatively include a graphic processing unit (GPU), such as for image processing, and/or an associated memory for the GPU. The GPU and/or the associated memory for the GPU may be interconnected via the system bus 1450 with the processor 1410, the memory 1420, the storage device 1430, and the input/output devices 1440. The memory associated with the GPU may store one or more images described herein, and the GPU may process one or more of the images described herein. The GPU may be coupled to and/or form a part of the processor 1410. The processor 1410 is capable of processing instructions for execution within the computing system 1400. Such executed instructions can implement one or more components of, for example, the deduplication engine 110. In some implementations of the current subject matter, the processor 1410 can be a single-threaded processor. Alternately, the processor 1410 can be a multi-threaded processor. The processor 1410 is capable of processing instructions stored in the memory 1420 and/or on the storage device 1430 to display graphical information for a user interface provided via the input/output device 1440.

The memory 1420 is a computer readable medium such as volatile or non-volatile that stores information within the computing system 1400. The memory 1420 can store data structures representing configuration object databases, for example. The storage device 1430 is capable of providing persistent storage for the computing system 1400. The storage device 1430 can be a floppy disk device, a hard disk device, an optical disk device, or a tape device, or other suitable persistent storage means. The input/output device 1440 provides input/output operations for the computing system 1400. In some implementations of the current subject matter, the input/output device 1440 includes a keyboard and/or pointing device. In various implementations, the input/output device 1440 includes a display unit for displaying graphical user interfaces.

According to some implementations of the current subject matter, the input/output device 1440 can provide input/output operations for a network device. For example, the input/output device 1440 can include Ethernet ports or other networking ports to communicate with one or more wired and/or wireless networks (e.g., a local area network (LAN), a wide area network (WAN), the Internet).

In some implementations of the current subject matter, the computing system 1400 can be used to execute various interactive computer software applications that can be used for organization, analysis and/or storage of data in various (e.g., tabular) format (e.g., Microsoft Excel®, and/or any other type of software). Alternatively, the computing system 1400 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1440. The user interface can be generated and presented to a user by the computing system 1400 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. For example, the logic flows may include different and/or additional operations than shown without departing from the scope of the present disclosure. One or more operations of the logic flows may be repeated and/or omitted without departing from the scope of the present disclosure. Other implementations may be within the scope of the following claims.

1. A computer-implemented method, comprising:
identifying, based at least on an identification information associated with a plurality of images depicting one or more slices of tissue from at least one block of a tissue sample, one or more of a duplicate image, a replicate image, and a multiple image present within the plurality of images;
identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and
updating the identification information associated with the plurality of images to indicate the one or more of the duplicate image, the replicate image, and the multiple image identified as present within the plurality of images.

2. The method of claim 1, wherein the identification information comprises at least a portion of a manifest, a scannable barcode, and/or metadata associated with the plurality of images.

3. The method of claim 1, wherein the identification information includes one or more of a sample identifier, a patient identifier, a block identifier, a slide identifier, an imaging modality, and a scanning parameter.

4. The method of claim 3, wherein a first image of the plurality of images is identified as a duplicate of a second image of the plurality of images based at least on the first image and the second image being associated with matching sample identifiers, patient identifiers, block identifiers, and slide identifiers.

5. The method of claim 3, wherein a first image of the plurality of images is identified as a replicate of a second image of the plurality of images based at least on the first image and the second image being associated with matching sample identifiers, patient identifiers, and block identifiers but different slide identifiers.

6. The method of claim 3, wherein a first image of the plurality of images is identified as a multiple of a second image of the plurality of images based at least on the first image and the second image being associated with matching sample identifiers and patient identifiers but different block identifiers and different slide identifiers.

7. The method of claim 1, wherein the identification information is updated by at least including, in the identification information, a flag indicating the one or more of the duplicate image, the replicate image, and the multiple image present within the plurality of images.

8. The method of claim 1, wherein the minimal difference between each pair of images corresponds to a minimal difference between a first vector field of a first image included in each pair of images and a second vector field of a second image included in each pair images.

9. The method of claim 8, further comprising:
generating the first vector field and the second vector field, each of the first vector field and the second vector field comprising a plurality of vectors, each vector of the plurality of vectors corresponding to a section within a corresponding image, and each vector of the plurality of vectors having a direction and a magnitude corresponding to a change in an intensity values of one or more pixels included a corresponding section of the corresponding image; and
determining the difference between the first vector field and the second vector field while adjusting the alignment between the first image and the second image until achieving the minimal difference between the first vector field and the second vector field.

10. The system of claim 9, further comprising:
dividing, into a plurality of sections, each of the first image and the second image; and
adjusting a size of each section of the plurality of sections and/or a quantity of the plurality of sections until the minimal difference between the first vector field and the second vector field is achieved.

11. The system of claim 1, wherein a first image in each pair of images is identified as a duplicate of a second image in each pair of images based at least on the metric satisfying a first threshold.

12. The system of claim 11, wherein the first image is identified as a replicate of the second image based at least on the metric failing to satisfy the first threshold but satisfying a second threshold greater than the first threshold.

13. The system of claim 12, further comprising:
identifying, based at least on the metric satisfying the second threshold, the first image and the second image as adjacent images from a same block of the tissue sample; and
updating the identification information associated with the plurality of images to indicate the first image and the second image as adjacent images from the same block of the tissue sample.

14. The system of claim 9, wherein the operations further comprise:
converting the first image and the second image into grayscale images prior to generating the first vector field and the second vector field.

15. The system of claim 9, further comprising:
converting, from a first scale to a second scale, a scale of pixel intensity values in each of the first image and the second image prior to generating the first vector field and the second vector field.

16. The system of claim 1, wherein the one or more of the duplicate image, the replicate image, and the multiple image present within the plurality of images are first identified based on the identification information before the metric is computed for a remaining plurality of images to identify the one or more of the duplicate image, the replicate images, and the multiple images in the remaining plurality of images.

17. The system of claim 1, further comprising:
updating the identification information to correct at least one discrepancy between the identification information and the one or more of the duplicate image, the replicate image, and the multiple image identified based on the metric.

18. The system of claim 1, further comprising:
updating a sequence of the plurality of images indicated in the identification information to restore, based at least on an ordering of at least one replicate image identified within the plurality of images, an original sequence of images within the at least one block of the tissue sample.

19. The system of claim 1, further comprising:
updating a sequence of the plurality of images indicated in the identification information to restore, based at least on an ordering of at least one multiple image identified within the plurality of images, an original sequence of images across different blocks of the tissue sample.

20. The system of claim 1, wherein the duplicate image depicts a same slice of tissue as another image included in the plurality of images, wherein the replicate image depicts a different slice of tissue from a same block of the tissue sample as the another image included in the plurality of images, and wherein the multiple image depicts a slice of tissue from a different block of the tissue sample as the another image from the plurality of images.

21. A system, comprising:
at least one processor; and
at least one memory storing instructions which, when executed by the at least one processor, result in operations comprising:
identifying, based at least on an identification information associated with a plurality of images depicting one or more slices of tissue from at least one block of a tissue sample, one or more of a duplicate image, a replicate image, and a multiple image present within the plurality of images;
identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and
updating the identification information associated with the plurality of images to indicate the one or more of the duplicate image, the replicate image, and the multiple image identified as present within the plurality of images.

22. A non-transitory computer readable medium storing instructions, which when executed by at least one processor, result in operations comprising:
identifying, based at least on an identification information associated with a plurality of images depicting one or more slices of tissue from at least one block of a tissue sample, one or more of a duplicate image, a replicate image, and a multiple image present within the plurality of images;
identifying, based at least on a metric computed for one or more pairs of images from the plurality of images, the one or more of the duplicate image, the replicate image, and the multiple image present within the plurality of images, the metric indicative of a minimal difference achieved between the each pair of images while adjusting an alignment therebetween; and
updating the identification information associated with the plurality of images to indicate the one or more of the duplicate image, the replicate image, and the multiple image identified as present within the plurality of images.
